(19) United States
(12) Patent Application Publication
(10) Pub. No.: US 2006/0095024 A1
(43) Pub. Date: May 4, 2006

(54) Photoablative laser with controllable pulse release frequency, and relative control method

(76) Inventor: Giuseppe D'Ippolito, Taranto (IT)

Correspondence Address: BLANK ROME LLP, 600 NEW HAMPSHIRE AVENUE, N.W., WASHINGTON, DC, 20037, US

(21) Appl. No.: 11/255,185

(22) Filed: Oct. 21, 2005

(30) Foreign Application Priority Data
Oct. 22, 2004 (IT) MI2004A 002020

(51) Int. Cl. A61B 18/18

(52) U.S. Cl. 606/5

(57) ABSTRACT

A photoablative laser includes an ablation profile memory device, in which are stored sets of coordinates defining a target volume for removal in the form of a number of layers of predetermined thickness and respective areas; and a laser pulse emission apparatus for sending laser pulses with a mean release frequency to the target volume to successively remove the layers. The photoablative laser also includes a control device associated with the laser pulse emission apparatus to control the mean release frequency of the laser pulses as a function of the respective areas of the layers, so that, when removing each layer, the target volume receives a number of laser pulses per unit of time and per unit of area below a predetermined threshold.

The present invention relates to a photoablative laser and relative control method.

### BACKGROUND OF THE INVENTION

As is known, photoablative lasers are commonly used in refractive surgery to reconstruct the cornea to correct visual defects, by removing successive layers of the cornea, varying in area, according to a predetermined ablation profile. Normally, the small-area layers are treated first, and then the larger-area layers. A photoablative laser sends pulse sequences of predetermined frequency and energy onto the cornea to locally evaporate microscopic volumes of cornea tissue. To avoid uneven ablation thickness caused by interaction between the laser spots striking the cornea and the cornea tissue evaporation fumes produced by the immediately preceding laser spots, and to prevent damage caused by overheating, the pulses are emitted to cover the layer for removal in a random as opposed to orderly sequence.

The commonly used method, however, is unsatisfactory, by only being effective as regards the large-area layers. When removing small-area layers of cornea tissue, the problems of uneven ablation thickness and overheating still remain, on account of energy accumulation still being considerable.

### SUMMARY OF THE INVENTION

It is an object of the present invention to eliminate the aforementioned drawbacks.

According to the present invention, there are provided a photoablative laser and a method of controlling a photoablative laser, as claimed in the attached Claims.

### BRIEF DESCRIPTION OF THE DRAWINGS

A number of non-limiting embodiments of the invention will be described by way of example with reference to the accompanying drawings, in which:

FIG. 1 shows a simplified block diagram of a photoablative laser in accordance with a first embodiment of the present invention;

FIG. 2 shows a more detailed block diagram of part of the photoablative laser in FIG. 1;

FIGS. 3 and 4 show diagrams of a patient's eye and a reference-axis system;

FIGS. 5 and 6 show simplified block diagrams of a second and third embodiment, respectively, of the present invention.

### DETAILED DESCRIPTION OF THE INVENTION

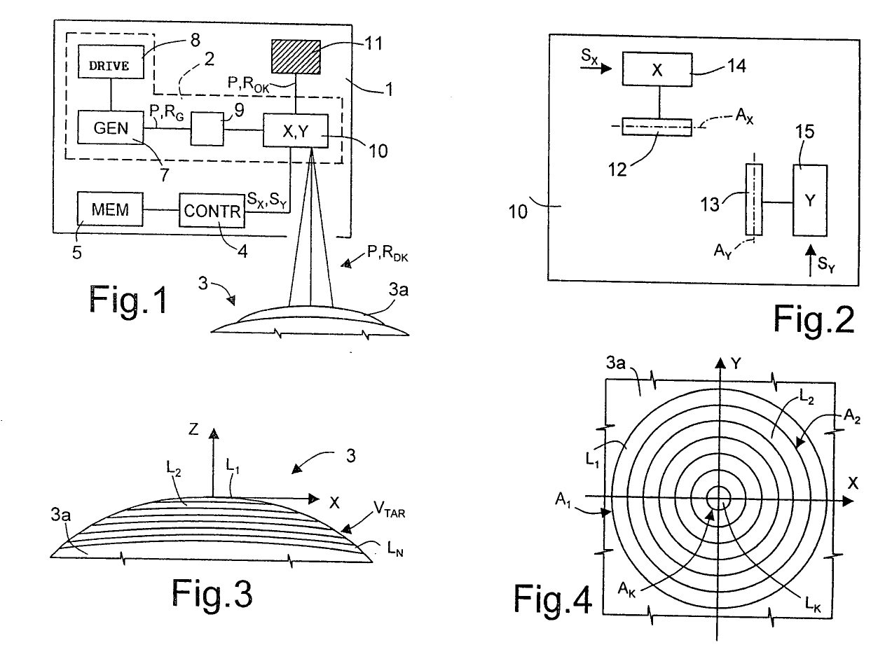

With reference to FIG. 1, a photoablative laser 1 for refractive surgery comprises an apparatus 2 for emitting laser pulses P to the cornea 3a of a patient's eye 3; a control device 4 associated with apparatus 2 to control emission of laser pulses P; and a memory device 5 storing an ablation profile as defined below.

Laser pulse emission apparatus 2 comprises a laser pulse generator 7 controlled by a drive unit 8; an optical system 9; a direction device 10; and an internal target 11.

Laser pulse generator 7 is a known, e.g. excimer or solid-state type, and supplies sequences of laser pulses P of predetermined energy with a generation frequency R_{G}, preferably of over 100 Hz.

Optical system 9, which is also known, is located along the path of laser pulses P, and may, for example, comprise collimators, lens systems, filters (not shown).

Direction device 10 intercepts laser pulses P and directs them as instructed by control device 4. As shown schematically in FIG. 2, direction device 10 comprises two mirrors 12, 13 located along the path of laser pulses P and orientable about respective perpendicular axes of rotation A_{X}, A_{Y} by means of actuators 14, 15 controlled by respective direction signals S_{X}, S_{Y} supplied by control device 4 (FIG. 1).

More specifically, control device 4 is connected to memory device 5, and generates direction signals S_{X}, S_{Y} on the basis of the ablation profile stored in the memory device.

The ablation profile is defined by sets of coordinates relative to a portion of cornea tissue 3a—hereinafter referred to as the target volume V_{TAR}—which must be removed to correct a refractive defect of eye 3. With reference to FIGS. 3 and 4, the coordinates are taken from a system of three perpendicular Cartesian axes X, Y, Z, wherein the Z axis coincides with the optical axis of eye 3. In the ablation profile stored in memory device 5, target volume V_{TAR} is defined in the form of a number of layers L₁, L₂, . . . , L_{N} for removal. Layers L₁, L₂, . . . , L_{N} are preferably of even thickness and respective areas A₁, A₂, . . . , A_{N}. In the FIG. 3 and 4 example, the selected ablation profile calls for removing the small-area layers L₁, L₂, . . . , L_{N} first. It should be pointed out that, in the case of a particularly uneven cornea 3a, some layers may include a number of non-connected regions; and the term “layers” may also include different portions of the same physical layer of cornea 3a to be removed at different stages and therefore stored separately in memory device 5.

Under the control of control device 5, emission apparatus 2 releases laser pulses P to target volume V_{TAR} with a mean release frequency R_{DK}. Here and hereinafter, the term “mean release frequency R_{DK}” refers solely to the mean frequency of the laser pulses P produced by laser pulse generator 7 and directed by direction device 10 to target volume V_{TAR} to remove a generic layer L_{K} in the time interval between commencing ablation of generic layer L_{K} and commencing removal of the next layer L₁, L₂, . . . , L_{N}. It is also understood that the duration of the step of removing generic layer L_{K} equals the duration of said time interval, and includes steps in which target volume V_{TAR} is reached by laser pulses P, and steps in which target volume V_{TAR} is not reached by laser pulses P. Mean release frequency R_{DK} is therefore less than or at most equal to generation frequency R_{G}.

Control device 4 controls direction device 10 in such a way as to direct laser pulses P alternately to target volume V_{TAR} and off target volume V_{TAR} (preferably to internal target 11), and to control mean release frequency R_{DK}. In fact, the greater the number of laser pulses diverted off target volume V_{TAR}, the lower the mean release frequency R_{DK}.

More specifically, mean release frequency R_{DK} is controlled as a function of areas A₁, A₂, . . . , A_{N} of layers L₁, L₂, . . . , L_{N}, so that, when removing each layer L₁, L₂, . . . , L_{N}, target volume V_{TAR} receives a number of laser pulses P, per unit of time and per unit of area, below a predetermined threshold N_{T}. In the embodiment of the invention described here, mean release frequency R_{DK} is set according to the equation:
R_{DK}=R_{G}*T_{DK}/T_{REF}=R_{G}*A_{K}/A_{REF} (1)

In (1), T_{REF} is the time taken to ablate a reference specimen layer of area A_{REF} greater than areas A₁, A₂, . . . , A_{N} of layers L₁, L₂, . . . , L_{N} at generation frequency R_{G}, to achieve a predetermined number N_{R}, below threshold N_{T}, of incident laser pulses P per unit of time and of cornea tissue area.
N_{R}≦N_{T} (2)

T_{DK} is the time taken to send the laser pulses P necessary to remove generic layer L_{K} of area A_{K}, and is given by the equation:
T_{DK}=T_{REF}*AK/A_{REF} (3)

In the embodiment described here, the number of laser pulses P striking target volume V_{TAR} per unit of time and per unit of area when removing each of layers L₁, L₂, . . . , L_{N} equals number N_{R}, is substantially constant, and is below predetermined threshold N_{T}.

Alternatively, number N_{R} may also vary, always below threshold N_{T}, as a function of area A₁, A₂, . . . , A_{N} of layers L₁, L₂, . . . , L_{N}. For example, number N_{R} may be slightly higher to remove layers L₁, L₂, . . . , L_{N} of smaller area A₁, A₂, . . . , A_{N}.

The frequency—indicated R_{OK}—with which direction device 10 diverts laser pulses P to internal target 11, on the other hand, is given by the equation:
R_{OK}=R_{G}−R_{DK} (4)

For each layer L_{K}, the total time T_{OK} the mirrors divert the laser spots onto internal target 11 equals
TOK=TREF−TDK (5)

When removing each layer L₁, L₂, . . . , L_{N}, total time T_{OK} may be an uninterrupted interval or an interval divided into a number of separate intervals.

In other words, control device 4 operates in such a way as to adapt mean release frequency R_{DK} to the respective areas A₁, A₂, . . . , A_{N} of layers L₁, L₂, . . . , L_{N} as they are removed.

The photoablative laser according to the invention has the advantage of preventing uneven ablation thickness caused by interaction between the laser pulses striking the cornea and the cornea tissue evaporation fumes produced by the immediately preceding laser pulses, and of preventing overheating of the cornea tissue during treatment and any possible damage this may cause. Obviously, maximum uniformity of ablation thickness is achieved using the same number of pulses per unit of time and area for all the layers.

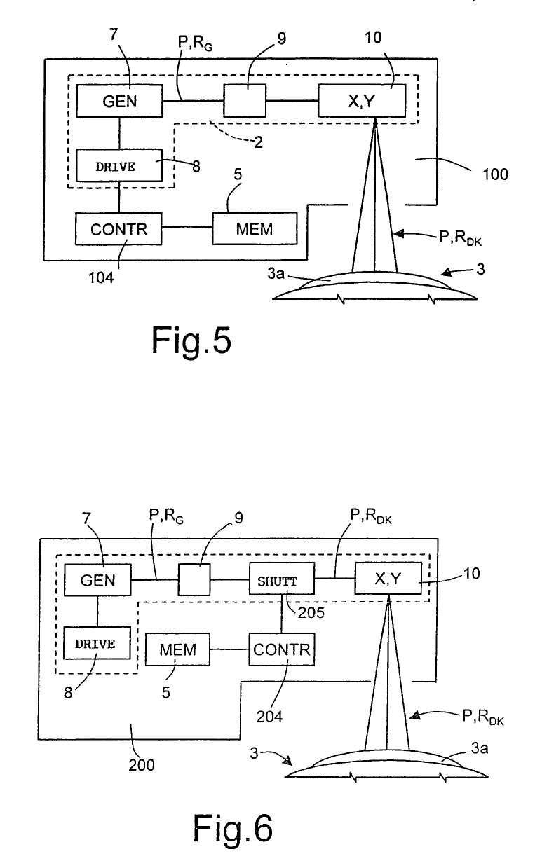

FIG. 5 shows a second embodiment of the invention, in which any parts identical to those already described are indicated using the same reference numbers. In this case, a photoablative laser 100 comprises apparatus 2 for emitting laser pulses P; a control device 104 associated with apparatus 2 to control emission of laser pulses P; and memory device 5.

Control device 104 is connected to the drive unit 8 of laser pulse generator 7, which emits laser pulses P at a variable generation frequency R_{G}. In other words, control device 104 acts on drive unit 8 to directly control generation frequency R_{G}, and to maintain the number N_{R} of laser pulses P sent to target volume V_{TAR} per unit of time and per unit of area below predetermined threshold N_{T}. Number N_{R} is preferably constant for all of layers L₁, L₂, . . . , L_{N}. In this case, mean release frequency R_{DK} equals generation frequency R_{G}. The generation frequency R_{G} values for each layer L₁, L₂, . . . , L_{N} are set in each individual case as described above, in particular with reference to equations (1)-(5).

In the FIG. 6 embodiment, the mean release frequency R_{DK} of a photoablative laser 200 is controlled by a control device 204, which modifies the activation time of a shutter 205 located along the path of laser pulses P.

Clearly, changes may be made to the method as described herein without, however, departing from the scope of the present invention as defined in the accompanying claims.

1. A photoablative laser comprising:
an ablation profile memory device, in which are stored sets of coordinates defining a target volume for removal in the form of a number of layers of predetermined thickness and respective areas a laser pulse emission apparatus for sending laser pulses with a mean release frequency to the target volume to remove said layers;
a control device associated with the laser pulse emission apparatus to control the mean release frequency of the laser pulses as a function of the respective areas of the layers so that when removing each layer, the target volume receives a number of laser pulses per unit of time and per unit of area below a predetermined threshold.

2. A laser as claimed in claim 1, wherein the number of pulses per unit of time and per unit of area is substantially equal for all the layers.

3. A laser as claimed in claim 2, wherein the laser pulse emission apparatus is controlled so as to prevent the laser pulses from being sent to the target volume for an uninterrupted time interval when removing each layer.

4. A laser as claimed in claim 2, wherein the laser pulse emission apparatus is controlled so as to prevent the laser pulses from being sent to the target volume for a number of separate time intervals when removing each layer.

5. A laser as claimed in claim 1, wherein the laser pulse emission apparatus comprises a laser pulse generating device supplying said laser pulses with a generation frequency; and a direction device for directing the laser pulses.

6. A laser as claimed in claim 5, wherein the control device is connected to the direction device to direct the laser pulses alternately to the target volume and off the target volume, so as to maintain below the predetermined threshold the number of laser pulses sent to the target volume per unit of time and per unit of area when removing each layer.

7. A laser as claimed in claim 5, wherein the direction device is controlled to reduce the mean release frequency to less than the generation frequency.

8. A laser as claimed in claim 5, wherein the direction device comprises two mirrors located along a path of said laser pulses and controlled by the control device.

9. A laser as claimed in claim 5, wherein the control device is connected to the laser pulse generating device to control the generation frequency, so that the number of laser pulses sent to the target volume per unit of time and per unit of area when removing each layer is maintained below the predetermined threshold.

10. A method of controlling a photoablative laser comprising the steps of:
defining a target volume for removal of a number of layers of predetermined thickness and respective areas;
sending laser pulses with a mean release frequency to the target volume to remove said layers; controlling the mean release frequency of the laser pulses as a function of the respective areas of the layers, so that, when removing each layer, the target volume receives a number of laser pulses per unit of time and per unit of area below a predetermined threshold.

11. A method as claimed in claim 10, wherein the number of pulses per unit of time and per unit of area is substantially equal for all the layers.

12. A method as claimed in claim 11, wherein the control step comprises preventing the laser pulses from being sent to the target volume for an uninterrupted time interval when removing each layer.

13. A method as claimed in claim 11, wherein the control step comprises preventing the laser pulses from being sent to the target volume for a number of separate time intervals when removing each layer.

14. A method as claimed in claim 10, further comprising the steps of generating laser pulses with a generation frequency; and directing the laser pulses.

15. A method as claimed in claim 14, wherein the laser pulses are directed alternately to the target volume and off the target volume, so as to maintain below the predetermined threshold the number of laser pulses (P) sent to the target volume per unit of time and per unit of area when removing each layer.

16. A method as claimed in claim 14, wherein the control step comprises reducing the mean release frequency to less than the generation frequency.

17. A method as claimed in claim 14, wherein the directing step comprises orienting two mirrors located along a path of the laser pulses.

18. A method as claimed in claim 14, wherein the step of controlling the release frequency comprises controlling the generation frequency, so that the number of laser pulses sent to the target volume per unit of time and per unit of area when removing each layer is maintained below the predetermined threshold.
